(19) United States
(12) Patent Application Publication
(10) Pub. No.: US 2023/0414135 A1
(43) Pub. Date: Dec. 28, 2023

(54) PATCH-TYPE BIOSENSOR

(71) Applicant: DONGWOO FINE-CHEM CO., LTD., Jeollabuk-do (KR)

(72) Inventors: Min-Soo YOO, Chungcheongnam-do (KR); Young-Keun LEE, Gyeonggi-do (KR); Soo-Ho CHO, Gyeonggi-do (KR); Seung-Hwan CHON, Gyeonggi-do (KR)

(21) Appl. No.: 18/265,024

(22) PCT Filed: Sep. 10, 2021
(86) PCT No.: PCT/KR2021/012357

§ 371 Date: Jun. 2, 2023

(30) Foreign Application Priority Data
Dec. 2, 2020 (KR) 10-2020-0166552

(51) Int. Cl. A61B 5/145; A61B 5/1477; B01L 3/00

(52) U.S. Cl. CPC A61B 5/14507; A61B 5/14546; A61B 5/14532; A61B 5/1477; B01L 3/502707; A61B 2560/0468; A61B 2562/164; A61B 2562/028; B01L 2300/0645; B01L 2300/0887

(57) ABSTRACT

Proposed is a biosensor including a first specimen inlet configured to provide a space through which a specimen flows inside, an electrode element configured to measure an electromechanical signal of the flowing-in specimen, a chamber configured to provide a space in which an electrochemical reaction of the flowing-in specimen occurs, and a first specimen outlet configured to provide a space through which the flowing-in specimen flows outside, in which a moisture absorber is disposed in the chamber.

### FIELD OF INVENTION

The present disclosure relates to a patch-type biosensor.

### BACKGROUND ART

A biosensor is a general term of devices or elements that can check existence or the amount of an analyte by reacting the analyte to be analyzed with a bio-receptor having a selectivity character and measuring the degree of the reaction through a signal transducer.

A biosensor is classified into an electrochemical sensor, a thermal sensor, an optical sensor, etc. in accordance with the transduction method, and recently, is called various names such as a glucose sensor, a cell sensor, an immuno biosensor, and a DNA chip, depending on the kinds of analytes to be analyzed.

An electrochemical sensor of those types is generally used up to now as a transduction method of a biosensor because it can convert the amount of biological specimen into an electrical signal that is easy to image-process.

Korean Patent No. 10-0887632 also provides a biosensor that is an electrochemical sensor using blood as a specimen and can accurately and conveniently measure various blood types without interference with them.

However, not only the biosensor of Korean Patent No. 10-0887632, but most biosensors of the related art analyze an analyte in the way of taking a specimen including an analyte to be analyzed from an analysis object, injecting the specimen into the sensors, and measuring an electrochemical signal. However, according to the way, there is a defect that it is required to artificially take a specimen from an analysis object and there is a defect that it is difficult to continuously measure an analyte included in a specimen because analysis of a specimen is temporarily performed. Further, in some cases, external air, etc. enter a biosensor together with a specimen when the specimen is put into the biosensor, so bubbles are contained in a biosensor other than a specimen that is a detection object, which causes a problem that accuracy is deteriorated in specimen detection.

Accordingly, it is required to develop a biosensor that can analyze an analyte by continuously obtaining a specimen even without artificially taking a specimen, improves reliability in measurement, and reduces measurement time. Further, it is required to develop a biosensor for solving problems such as deterioration of detection accuracy due to bubbles produced in a biosensor.

### DISCLOSURE

### Technical Problem

An objective of the present disclosure is to provide a patch-type biosensor.

Another objective of the present disclosure is to provide a biosensor that can perform continuous measurement through continuous inflow and outflow of a specimen.

Another objective of the present disclosure is to provide a biosensor for minimizing differences between measurement samples due to bubbles that are produced in flow passages when a specimen flows inside and outside.

### Technical Solution

The present disclosure relates to a biosensor including a first specimen inlet configured to provide a space through which a specimen flows inside, an electrode element configured to measure an electromechanical signal of the flowing-in specimen, a chamber configured to provide a space in which an electrochemical reaction of the flowing-in specimen occurs, and a first specimen outlet configured to provide a space through which the flowing-in specimen flows outside, in which a moisture absorber is disposed in the chamber.

In a first aspect of the present disclosure, the moisture absorber may have a porosity of 0.5 to 0.8 that is calculated by following Equation 1,

$$ε = 1 - \frac{{bw}_{0}}{{ρ}_{cel} {τ}_{p}} \qquad [ Equation 1 ]$$

In the Equation 1,

ε is a porosity of the moisture absorber, bw₀ is basis weight (kg/m²) of the moisture absorber, ρ_{cel} is density of cellulose (kg/m³) of the moisture absorber, and τₚ is a thickness (m) of the moisture absorber.

In a second aspect of the present disclosure, a height of the chamber may be 50 to 1,000 μm.

In a third aspect of the present disclosure, the biosensor may have a stacked structure including: a first base, a second base formed on the first base; and a third base formed on the second base.

In a fourth aspect of the present disclosure, the first specimen inlet may be disposed in the first base.

In a fifth aspect of the present disclosure, a width of the first specimen inlet may be 100 to 1,0001 μm.

In a sixth aspect of the present disclosure, the chamber may be disposed in the second base.

In a seventh aspect of the present disclosure, the second base may further include: a second specimen inlet formed at a position corresponding to the first specimen inlet; and a channel configured to guide a specimen flowing in the second specimen inlet to the chamber.

In an eighth aspect of the present disclosure, a height of the channel may be 100 to 1,000 μm.

In a ninth aspect of the present disclosure, the chamber may be directly connected to the first specimen inlet.

In a tenth aspect of the present disclosure, the electrode element may be disposed between the first base and the second base.

In an eleventh aspect of the present disclosure, the first base and the second base each may independently include one or more kinds selected from a group of glass, polyethersulfone (PES), poly methyl methacrylate (PMMA), polycarbonate (PC), polyethylene (PE), polyethylene naphthalate (PEN), polyphenylene sulfide (PPS), polypropylene (PP), triacetyl cellulose (TAC), cellulose acetate propionate (CAP), polyethylene terephthalate (PET), polyimide (PI), polyetherimide (PEI), polyamide (PA), cyclo olefin polymer (COP), cyclo olefin copolymer (COC), PMMA/PC copolymer, and PMMA/PC/PMMA copolymer.

In a twelfth embodiment of the present disclosure, the second base may be made of a PSA (pressure sensitive adhesive) compound or an OCA (optical clear adhesive) compound.

In a thirteenth aspect of the present disclosure, the biosensor may further include a fourth base formed under the first base and the fourth base may have a third specimen inlet.

In a fourteenth aspect of the present disclosure, the first specimen outlet may be disposed in the third base.

In a fifteenth aspect of the present disclosure, a width of the first specimen outlet may be 100 to 1,000 μm.

### Advantageous Effects

Since a biosensor according to the present disclosure suppresses bubbles that may be produced in a chamber when a specimen flows inside by having a moisture absorber, which easily absorbs moisture, in a chamber, differences between samples are minimized, whereby it is possible to improve detection precision and reduce a measurement time.

Further, since a biosensor according to the present disclosure can smoothly obtain a specimen even without a specific device by appropriately adjusting the number, width, etc. of bases, specimen inlets, and specimen outlets, it is possible to remove the inconvenience of artificially taking a specimen from an analysis object.

Further, a biosensor according to the present disclosure can continuously measure an analyte included in a specimen by continuous inflow and outflow of a specimen.

### DESCRIPTION OF DRAWINGS

FIG. 1 is an exploded perspective view showing a biosensor that is an embodiment of the present disclosure.

FIG. 2 is a cross-sectional view of the biosensor of FIG. 1.

FIG. 3 is a perspective view showing a first base included in a biosensor that is one or a plurality of embodiments of the present disclosure.

FIG. 4 is a perspective view showing a second base included in a biosensor that is one or a plurality of embodiments of the present disclosure.

FIG. 5 is a perspective view showing a third base included in a biosensor that is an embodiment of the present disclosure.

FIG. 6 is a perspective view showing a fourth base included in a biosensor that is one or a plurality of embodiments of the present disclosure.

FIG. 7 is a view showing stabilization index evaluation results of biosensors according to a third embodiment of the present disclosure and a first comparative example.

In these figures, the meanings of reference numerals are as follows.
10: first base
11: first specimen inlet
12: working electrode
13: reference electrode
20: second base
21: second specimen inlet
22: chamber
23: channel
24: second specimen outlet
25: moisture absorber
30: third base
31: first specimen outlet
40: fourth base
41: third specimen inlet

### BEST MODE

The present disclosure relates to a patch-type biosensor derived from the fact that it is possible to continuously measure an analyte included in a specimen because a specimen continuously flows inside and outside by pressure generated by the specimen even without artificially taking a specimen from an analyte when manufacturing a biosensor in a patch type.

In particular, the present disclosure relates to a biosensor for reducing differences between measurement samples by minimizing production of bubbles that may be produced in flow passages when a specimen flows inside and outside.

In detail, a biosensor of the present disclosure may include: a first specimen inlet configured to provide a space through which a specimen flows inside; an electrode element configured to measure an electromechanical signal of the flowing-in specimen; a chamber configured to provide a space in which an electrochemical reaction of the flowing-in specimen occurs; and a first specimen outlet configured to provide a space through which the flowing-in specimen flows outside, in which a moisture absorber is disposed in the chamber.

As described above, since a moisture absorber is disposed in the patch-type biosensor having a microfluidics structure, fluidity of a specimen is secured, whereby it is possible to enable a specimen to smoothly flow into the biosensor and it is possible to improve reliability in measurement even using only a small amount of specimen, as compared with biosensors in the related art, by suppressing production of bubbles in flow passages.

Hereafter, embodiments of the present disclosure are described in more detail with reference to drawings. However, the accompanying drawings of this specification exemplify preferred embodiments and help easy understanding of the present disclosure together with the present disclosure described above, so the present disclosure should not be construed as being limited to those in the drawings.

The terms used herein are provided to describe embodiments without limiting the present disclosure. In the specification, a singular form includes a plural form unless specifically stated in the sentences.

The terms “comprises” and/or “comprising” used herein do not exclude that one or more other components, steps, operations, and/or elements exist or are added other than the stated component, step, operation, and/or element. Like reference numerals indicate like components throughout the specification.

Spatial relative terms “below”, “beneath”, “lower”, “above”, “upper”, etc. may be used to easily describe the correlation of one element of component and another element or component, as shown in the drawings. The spatially relative terms should be construed as terminal including different directions of elements in using or in operating in addition to the directions shown in figures. For example, when elements shown in the drawings are turned upside down, an element described as being “below” or “beneath” another element may be positioned “over” the another element. Accordingly, “below” and “beneath” that are exemplary terms may include both of up and down directions. An element may be oriented in different directions, so the spatially relative terms may be construed in accordance with orientation.

<Biosensor>

A biosensor of the present disclosure may be for guiding smooth movement of a specimen and suppressing production of bubbles in flow passages by including a moisture absorber therein. In detail, the biosensor may include: a first specimen inlet into which a specimen flows; an electrode that generates an electrochemical reaction; a chamber that provides a space for a reaction; and a first specimen outlet that guides outflow of a flowing-in specimen, in which a moisture absorber may be disposed in the chamber.

Further, the present disclosure may be formed in a stacked structure in terms of ease in manufacturing, economics in process, etc. In detail, the present disclosure may include a first base, a second base formed on the first base, and a third base formed on the second base.

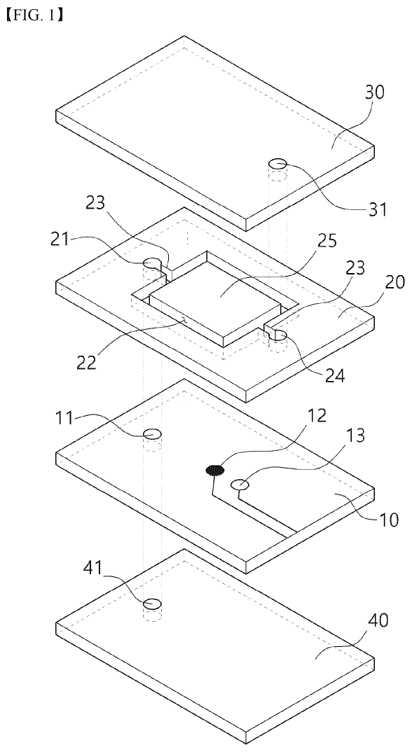

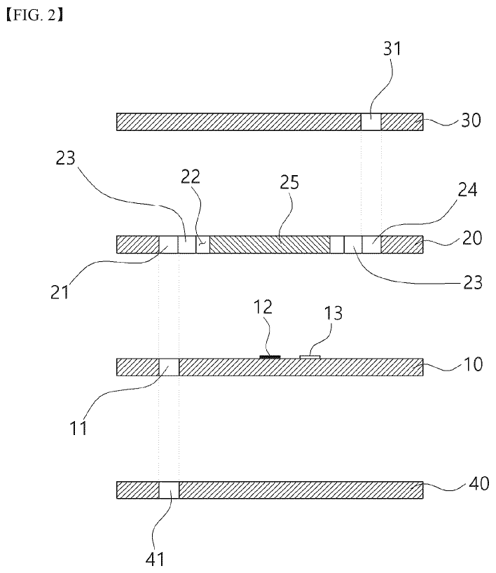

FIG. 1 is an exploded perspective view showing a biosensor that is an embodiment of the present disclosure. FIG. 2 is a cross-sectional view of the biosensor shown in FIG. 1.

Referring to FIGS. 1 and 2, a biosensor may have a stacked structure including a first base 10, a second base 20 formed on the first base 10, and a third base 30 formed on the second base 20. Further, the biosensor may include a first specimen inlet 11 for providing a space through which a specimen flows inside, electrode elements 12 and 13 for measuring an electromechanical signal of the flowing-in specimen, a chamber 22 for providing a space for an electrochemical reaction of the flowing-in specimen and the electrode elements 12 and 13, and a first specimen outlet 31 for providing a space through which the flowing-in specimen flows outside, and may include a moisture absorber 25 disposed in the chamber 22.

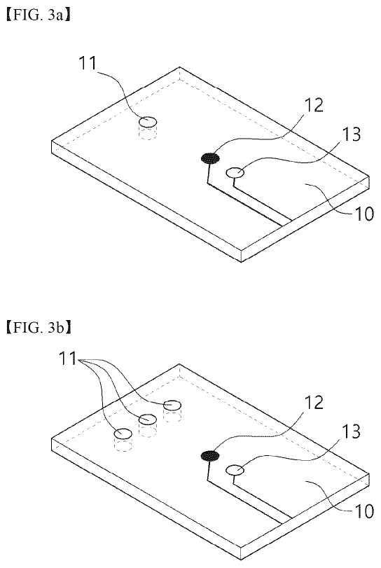

FIG. 3 is a perspective view showing the first base 10 included in a biosensor according to exemplary embodiments.

In one or a plurality of embodiments, the thickness of the first base 10 may be 100 to 1,000 μm.

Referring to FIG. 3, the first base 10 may include the first specimen inlet 11 formed on the bottom of the first base 10 to pass through the first base 10.

The number of the first specimen inlet 11 is not specifically limited as long as it enables a specimen smoothly to flow inside, and in an embodiment, a single first specimen inlet may be provided, as shown in FIG. 3a. Further, in some embodiments, a plurality of first specimen inlets 11 may be provided, and for example, as shown in FIG. 3b, three first specimen inlets 11 may be included. In this case, when a specimen flows and moves in the biosensor, bubbles may not be produced and the specimen can quickly flow into the chamber 22.

In one or a plurality of embodiments, the width of the first specimen inlet 11 may be 100 to 1,000 μm, preferably may be 150 to 600 μm, and more preferably may be 200 to 400 μm. When the width of the first specimen inlet 11 satisfies this range, a specimen secreted from an analyte smoothly flows into and moves in the biosensor by the pressure of the specimen even without a specific device, so bubbles may not be produced in the biosensor.

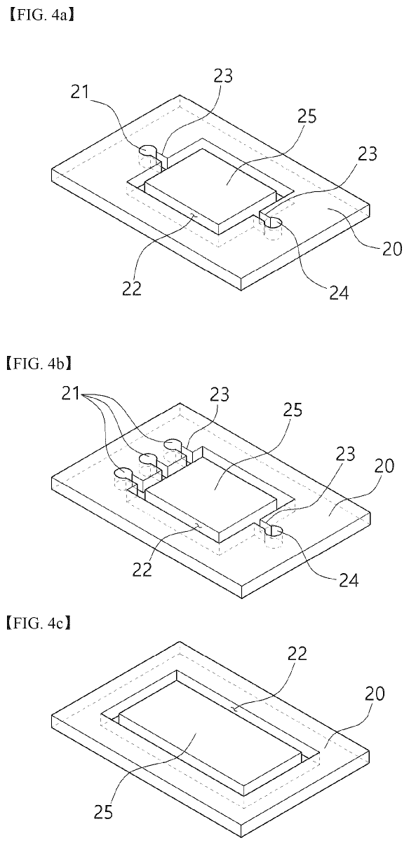

FIG. 4 is a perspective view showing the second base 20 included in a biosensor according to exemplary embodiments.

In an embodiment, the second base 20 may be provided as an adhesive surface positioned between the first base 10 and the third base 30, for example, may be an adhesive, etc., and preferably may be made of a PSA (pressure sensitive adhesive) compound or an OCA (optical clear adhesive) compound.

In an embodiment, the second base 20 may be provided as a base layer in which the chamber 22 is formed.

The chamber 22 may be provided to provide a space in which a flowing-in specimen generates an electrochemical reaction with the electrode elements 12 and 13.

In an embodiment, the height of the chamber 22 may be 50 to 1,000 μm, preferably may be 50 to 500 μm, and more preferably may be 100 to 300 μm. When the height of the chamber 22 satisfies this range, it is possible to prevent reduction of the speed of a specimen filling the chamber 22, reduce the minimum necessary amount of a specimen for measurement, and suppress production of bubbles while the chamber is filled with a specimen.

The chamber 22, in an embodiment, as shown in FIG. 4a, has a second specimen inlet 21 and a second specimen inlet 24, and the second specimen inlet 21 and the second specimen inlet 24 may be connected by a channel 23.

In one or a plurality of embodiments, the width of the second specimen inlet 21 may be 100 to 1,000 μm, preferably may be 150 to 600 μm, and more preferably may be 200 to 400 μm. When the width of the second specimen inlet 21 satisfies this range, a specimen smoothly flows inside and moves, so bubbles may not be produced when a specimen flows into and move in the biosensor.

The second specimen inlet 21, preferably, may be formed at a position corresponding to the first specimen inlet 11 and provided as a space into which a specimen supplied from the first specimen inlet 11 flows.

In an embodiment, as shown in FIG. 4a, a single second specimen inlet 21 corresponding to the single first specimen inlet 11 may be included.

In some embodiment, a plurality of second specimen inlets 21 may be provided, and for example, as shown in FIG. 4b, three second specimen inlets 21 may be included in correspondence to the three first specimen inlets 11 formed in the first base 10. In this case, since a specimen is supplied from the plurality of first specimen inlets 11, it is possible to quickly supply a specimen to the chamber 22, and bubbles may not be produced when a specimen flows into and moves in the chamber 22.

The channel 23 may be provided as a guide that guides the a specimen supplied from the second specimen inlet 21 to the chamber 22 and guides the specimen flowing out of the chamber 22 to the second specimen outlet 24.

In one or a plurality of embodiments, the width of the channel 23 may be 100 to 1,000 μm, preferably may be 150 to 600 μm, and more preferably may be 200 to 400 μm. When the width of the channel 23 satisfies this range, a specimen smoothly moves, so bubbles may not be produced when a specimen moves in the biosensor.

The second specimen outlet 24 may be provided as a space into which the specimen flowing out of the chamber 22 is guided by the channel 23.

In an embodiment, the second specimen outlet 24, as shown in FIG. 4a and FIG. 4b, may include a single second specimen outlet 24. However, the number of the second specimen outlet 24 is not specifically limited and can be appropriately selected by a user to appropriately adjust inflow and outflow of a specimen, so a plurality of second specimen outlets 24 may be included.

In one or a plurality of embodiments, the width of the second specimen outlet 24 may be 100 to 1,000 μm, preferably may be 150 to 600 μm, and more preferably may be 200 to 400 μm. When the width of the second specimen outlet 24 satisfies this range, a specimen smoothly flows into and out of the chamber 22, so bubbles may not be produced when a specimen moves in the biosensor.

In some embodiments, the chamber 22, as shown in FIG. 4c, may be formed in an integrated type without the second specimen inlet 21, the second specimen outlet 24, and the channel 23. In this case, the first specimen inlet 11 is directly connected to the chamber 22 to supply a specimen to the chamber 22.

Further, referring to FIGS. 4a to 4c, the chamber 22 may have a moisture absorber 25 to induce smooth movement of a specimen.

The moisture absorber 25 is not specifically limited as long as it can induce smooth movement of a specimen and suppress production of bubbles in flow passages. In one or a plurality of embodiments, the moisture absorber may be a filter paper including α-Cellulose, etc. and can filter out particles at the level of micrometer (μm), and, depending on cases, may contain ash of 0.005 to 0.1%. As products on the market, Whatman® Grade 1 Qualitative Filter Paper, Whatman® Grade 2 Qualitative Filter Paper, Whatman® Grade 4 Qualitative Filter Paper, Whatman® Grade 6 Qualitative Filter Paper, etc. by Whatman may be used.

It is preferable that the moisture absorber 25 is selected in consideration of the porosity thereof to suppress production of bubbles in flow passages and guide smooth movement of a specimen. In detail, the porosity of the moisture absorber 25 that is calculated by the following Equation 1 may be preferably 0.5 to 0.8, and more preferably 0.6 to 0.75.

$$ε = 1 - \frac{{bw}_{0}}{{ρ}_{cel} {τ}_{p}} \qquad [ Equation 1 ]$$

In Equation 1,

ε is the porosity of the moisture absorber, bw₀ is the basis weight (kg/m²) of the moisture absorber, ρ_{cel} is the density of cellulose (kg/m³) of the moisture absorber, and τₚ is the thickness (m) of the moisture absorber.

Further, in another embodiment, the product of the porosity calculated by Equation 1 and the thickness of the moisture absorber 25 may be preferably 95 μm to 160 μm, and more preferably may be 95 μm to 150 μm.

When a porosity and/or the product of the porosity and the thickness of moisture absorber 25 satisfies this range, fluidity of a specimen is further improved and production of bubbles in flow passages can be more efficiently suppressed, so it is possible to not only reduce data distribution of measurement samples, but decrease the time that is taken for measurement.

Meanwhile, a porosity is calculated by generally considering several parameters including not only a pore size, but pore density, etc., and it is apparent to those skilled in the art that a porosity cannot be expected from only a specific parameter and should be calculated generally in consideration of several parameters. For example, even though a pore size increases, porosity may decrease, and even though pore density decreases, porosity may increase. In one or a plurality of embodiments, it is preferable that the pore size of the moisture absorber 25 is 1 to 15 μm in respect that smooth movement of a specimen can be guided and it is possible to suppress production of bubbles, but when the range of the porosity that is calculated by Equation 1 is not satisfied even though the pore size satisfies the range described above, the effect of improving fluidity of a specimen and suppressing production of bubbles may be deteriorated.

The area of the moisture absorber 25 is not specifically limited as long as it can guide smooth movement of a specimen and suppress production of bubbles in flow passages, but it is preferable that at least the electrode elements 12 and 13 are included in respect of reducing data distribution of measurement samples and reduction of measurement time.

In one or a plurality of embodiments, the thickness of the moisture absorber 25 may be 100 to 1,000 μm, preferably may be 100 to 500 μm, and more preferably may be 150 to 350 μm. When the thickness of the moisture absorber satisfies this range, it is possible to maintain porosity at an appropriate level, which is advantageous in terms of fluidity improvement of a specimen and bubble production suppression.

The biosensor of the present disclosure may include a first electrode element 12 and a second electrode element 13 configuring the electrode elements 12 and 13 for measuring an electrical signal by reaction of a specimen.

In an embodiment, the first electrode element 12 and the second electrode element 13 may be formed on the top of the first base 10, and preferably, may be formed on the top of the region of the first base 10 that corresponds to the region in which the chamber 22 is formed in the second base 20.

In an embodiment, the first electrode element 12 may be a working electrode and the second electrode element 13 may be a reference electrode.

The first electrode element 12 configuring a working electrode is an electrode that reacts with a specimen, and may be provided as an electrode that makes a current flow in an electrode reaction.

In one or a plurality of embodiments, one or more kinds selected from a group of gold (Au), silver (Ag), copper (Cu), platinum (Pt), titanium (Ti), nickel (Ni), tin (Sn), molybdenum (Mo), palladium (Pd), cobalt (Co), an alloy thereof, pyrolytic graphite, glassy carbon, carbon paste, PFC (perfluoro carbon), and CNT (carbon nanotube) may be used for the first electrode element 21 configuring a working electrode, but carbon paste is preferable, considering ease in manufacturing, excellence in implementation, and a potential window of a wide oxidation/reduction direction. These substances may be independently used, but the present disclosure is not limited thereto and the first electrode element may be used as a multilayer film of two or more materials.

The second electrode element 13 configuring a reference electrode may be provided as an electrode that has constant potential and is a reference for obtaining evolution electrode of a working electrode.

In one or a plurality of embodiments, one or more kinds selected from a group of a silver-silver chloride (Ag/AgCl) electrode, a calomel electrode, a mercury-mercury sulfate electrode, a mercury-oxide mercury electrode, etc. may be used as the second electrode element 13 configuring a reference electrode, and a silver-silver chloride (Ag/AgCl) electrode is preferable, considering that potential is stable up to high temperatures.

In some embodiments, in addition to the first electrode element 12 and the third electrode element 13, a third electrode element (not shown) and an electrode protective layer may be further included.

The third electrode element may be a count electrode and may be provided as an electrode that sends out or receives a current such that a reaction is generated on the surface of a working electrode.

In one or a plurality of embodiments, all of the materials described for the first electrode element 12 and the second electrode element 13 may be used for the third electrode element configuring a count electrode, and it is preferable to use the same materials as those of the first electrode element 12 and/or the second electrode element 13 in order to simplifying the process and reducing the manufacturing cost.

A working electrode configuring the first electrode element 12, a reference electrode configuring the second electrode element 13, and a count electrode configuring the third electrode may be manufactured by a common manufacturing method. In one or a plurality of embodiments, one or more processes selected from a group of screen printing, letterpress printing, intaglio printing, planography, and photolithography may be included. In one embodiment, it is preferable to form the electrodes integrally with wirings by performing photolithography, and the electrodes may be manufactured by any one selected from a group of screen printing, letterpress printing, intaglio printing, and planography, and screen printing may be preferable.

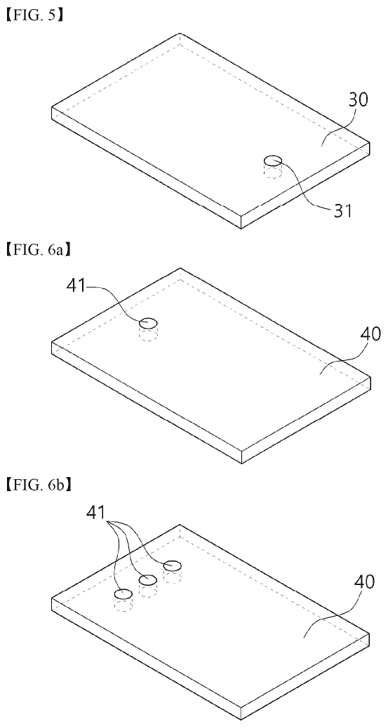

FIG. 5 is a perspective view showing the third base 30 included in a biosensor according to exemplary embodiments.

In one embodiment, the third base 30 shields the second specimen inlet 21, the chamber 22, the channel 23, the second specimen outlet 24, etc. formed in the second base 20 from the outside and may be provided as a cover of the biosensor.

In one or a plurality of embodiments, the thickness of the third base 30 may be 100 to 1,0001 μm.

Referring to FIG. 5, the third base 30 may include a first specimen outlet 31 formed on the bottom of the third base 30 to pass through the third base 30.

In one embodiment, the first specimen outlet 31 is formed as a position corresponding to the second specimen outlet 24 formed in the second base 20 and may be provided as a passage through which the specimen flowing out of the second specimen outlet 24 flows outside.

In one or a plurality of embodiments, the width of the first specimen outlet 31 may be 100 to 1,000 μm, preferably may be 150 to 600 μm, and more preferably may be 200 to 400 μm. When the width of the first specimen outlet 31 satisfies this range, a specimen smoothly flows into and out of the biosensor, so bubbles may not be produced.

In an embodiment, as shown in FIG. 5, a single first specimen outlet 31 corresponding to the single second specimen outlet 24 may be included. However, the number of the first specimen outlet 31 is not specifically limited and can be appropriately selected by a user to appropriately adjust inflow and outflow of a specimen, so a plurality of first specimen outlets 31 corresponding to the plurality of second specimen outlets 24 of the second base 20 may be included.

In one or a plurality of embodiments, the first base 10 and the third base 30 are not specifically limited, and for example, each may independently include one or more kinds selected from a group of glass, polyethersulfone (PES), poly methyl methacrylate (PMMA), polycarbonate (PC), polyethylene (PE), polyethylene naphthalate (PEN), polyphenylene sulfide (PPS), polypropylene (PP), triacetyl cellulose (TAC), cellulose acetate propionate (CAP), polyethylene terephthalate (PET), polyimide (PI), polyetherimide (PEI), polyamide (PA), cyclo olefin polymer (COP), cyclo olefin copolymer (COC), PMMA/PC copolymer, and PMMA/PC/PMMA copolymer.

In one embodiment, the first base 10 and the third base 30 may be manufactured using the same material, and in this case, it is possible to simplify the process and reduce the manufacturing cost.

In some embodiments, the biosensor may further include a fourth base 40 on the bottom of the first base 10.

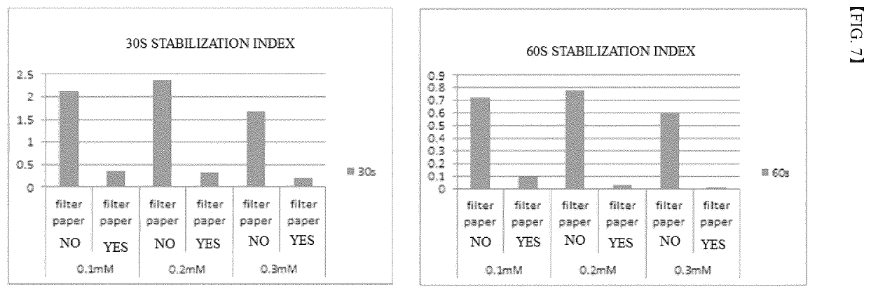

FIG. 6 is a perspective view showing the fourth base 40 included in a biosensor according to exemplary embodiments.

In one or a plurality of embodiments, the thickness of the fourth base 40 may be 50 to 1,000 μm.

In an embodiment, the fourth base 40 may be provided as an adhesive surface positioned between a patch-type biosensor and an analysis object, for example, may be an adhesive, etc., and preferably may be produced from a PSA (pressure sensitive adhesive) compound or an OCA (optical clear adhesive) compound.

In an embodiment, the fourth base 40 has a third specimen inlet 41 formed at a position corresponding to the first specimen inlet 11 formed on the bottom of the first base 10, whereby it may be provided as a guide for guiding a specimen coming from an analysis object to the first specimen inlet 11 through the third specimen inlet 41.

In an embodiment, as shown in FIG. 6a, a single third specimen inlet 41 corresponding to the single first specimen inlet 11 may be included.

In some embodiment, a plurality of third specimen inlets 41 may be provided, and for example, as shown in FIG. 6b, three third specimen inlets 41 may be included in correspondence to the three first specimen inlets 11 formed in the first base 10. In this case, since a specimen is guided to the plurality of first specimen inlets 11, when a specimen flows into and moves in the biosensor, bubbles may not be produced and the specimen can quickly flow into the chamber 22.

In one or a plurality of embodiments, the width of the third specimen inlet 41 may be 100 to 3,000 μm.

In one or a plurality of embodiments, a specimen including an analyte to be analyzed may be a liquid specimen and, for example, may be a biological sample such as blood, body fluid, urine, saliva, and sweat, but is not limited thereto.

In one or a plurality of embodiments, an analyte to be analyzed, for example, may be glucose, lactate, cholesterol, vitamin C (ascorbic acid), alcohol, various cations, and various anions, but is not limited thereto.

<Method of Measuring Electrochemical Signal>

The present disclosure includes a method of measuring an electrochemical signal of an analyte included in a specimen using the biosensor. According to the method of measuring an electrochemical signal of the present disclosure, it is possible to obtain a specimen even though not artificially taking a specimen from an analysis object, and it is possible to continuously measure an analyte included in a specimen by continuous inflow and outflow of a specimen.

This results from a microfluidics structure that uses pressure that is generated when a specimen is secreted from an analysis object, and, unlike capillary action that occurs even in capillaries without a specific limit, may be achieved by appropriately adjusting the number, width, thickness, etc. of the bases, specimen inlets, and specimen outlets described in the above item <Biosensor>.

In this specification, [symbol] electrochemically measure [symbol] , means measuring by applying an electrochemical measurement technique. In one or a plurality of embodiments, amperometry, potentiometry, culometry, etc. may be exemplified, and potentiometry may be preferable.

Hereafter, the method of measuring an electrochemical signal of an analyte is described in more detail with reference to drawings. However, it was described above that the method is not construed as being limited to those in the drawings.

Referring to FIGS. 1 and 2, the fourth base 40 configuring the lowermost layer of the patch-type biosensor may be attached to an analysis object and is preferably attached to the point where a specimen is secreted. In an embodiment, the biosensor of the present disclosure, which is for measuring glucose included in sweat, may be attached to an upper arm.

By the pressure of a specimen secreted from an analysis object, a portion of the specimen is guided to the first specimen inlet 11 of the first base 10 through the third specimen inlet 41 on the bottom of the fourth base 40.

The specimen guided into the first specimen inlet 11 is guided to the second specimen inlet 21 formed in the second base 20 and then guided by the channel 23, thereby moving into the chamber 22.

The specimen that has moved into the chamber 22 moves toward the second specimen outlet 24 while filling the chamber 22 through the moisture absorber 25 disposed in the chamber 22. In this process, the analyte included in the specimen reacts with a receptor formed at the first electrode element 21 configuring a working electrode, thereby generating electrical variation.

The method measures a response current value discharged in correspondence to the electrical variation by applying a voltage to an electrode element including the first electrode element 12 and the second electrode element 13, and derives an electrochemical signal of an analyte in the specimen on the basis of the response current value.

The voltage that is applied is not specifically limited, but, in one or a plurality of embodiments, the voltage may be −500 to +500 mV, and preferably, −200 to +200 mV for a silver-silver chloride electrode (Ag/AgCl electrode).

The method of measuring an electrochemical signal of a detection object, in other embodiments, may apply a voltage to the electrode element without applying a voltage for a predetermined time after contact with the reagent or may apply a voltage to the electrode element simultaneously with contact with the reagent.

Thereafter, the specimen that finishes reacting with the first electrode element 12 is guided to the second specimen outlet 24 by the channel 23 and then flows outside through the first specimen outlet 31 formed in the third base 30.

According to the biosensor of the present disclosure, the series of processes described above is not temporarily performed and a specimen secreted from an analysis object continuously flows inside and outside by the pressure of the specimen, so it is possible to continuously measure an analyte included in a specimen. Further, since a specimen moves through the moisture absorber, production of bubbles in flow passages, particularly, the chamber, in which an electrochemical reaction with the electrode element is generated, is suppressed, so it is possible to improve reliability in measurement even using only a small amount of specimen.

<System for Measuring Electrochemical Signal>

The present disclosure includes a system for measuring an electrochemical signal, the system including the biosensor, a component that applies a voltage to the electrode elements of the biosensor, and a component that measures a current at the electrode elements, and measuring an electrochemical signal of an analyte in a specimen. According to the method of measuring an electrochemical signal of the present disclosure, it is possible to obtain a specimen even though not artificially taking a specimen from an analysis object, and it is possible to continuously measure an analyte included in a specimen by continuous inflow and outflow of a specimen.

The component that applies a voltage is not specifically limited as long as it is electrically connected to the electrode elements of the biosensor and can apply a voltage, and well-known voltage-applying component can be used. The component that applies a voltage, in one or a plurality of embodiments, may include a contactor that can come in contact with the electrode elements of the biosensor, a power source such as a DC power source, etc.

The component that measures an analyte, which is for measuring a plurality of currents generated at the electrode elements when a voltage is applied, in one or a plurality of embodiments, has only to be able to measure a response current value related to the amount of electrons discharged from the electrode elements of the biosensor, and components, which are used in biosensors that have been or will be developed, may be used.

### MODE FOR INVENTION

Hereafter, preferred embodiments of the present disclosure are described in detail. However, the present disclosure is not limited to the embodiments to be described hereafter and may be implemented in various ways, the embodiments are provided to complete the description of the present disclosure and let those skilled in the art completely know the scope of the present disclosure, and the present disclosure is defined by claims.

### EXAMPLES AND COMPARATIVE EXAMPLES

Biosensors according to Examples and comparative examples were manufactured with reference to the contents in the following Tables 1 and 2.

### EXAMPLES

A first specimen inlet for guiding inflow of a specimen was formed in a PET film configuring a first base using a laser cutter. Thereafter, a working electrode and a reference electrode were printed by screen printing to correspond to the position of a chamber in a second base using carbon paste and silver paste, respectively.

A second specimen inlet, a chamber, and a second specimen outlet were formed in an OCA film, which configures the second base, in the same way as the above. Thereafter, a moisture absorber was arranged to correspond to the chamber.

A first specimen outlet was formed in a PET film, which configures a third base, in the same way as the above.

A third specimen inlet was formed in an OCA film, which configures a fourth base, in the same way as the above.

The specimen inlet and the specimen outlet formed in each of the first to fourth bases are attached and stacked to correspond to each other, thereby biosensors of first to fourth Examples were manufactured.

### COMPARATIVE EXAMPLES

A first specimen inlet for guiding inflow of a specimen was formed in a PET film configuring a first base using a laser cutter. Thereafter, a working electrode and a reference electrode were printed by screen printing to correspond to the position of a chamber in a second base using carbon paste and silver paste, respectively.

A second specimen inlet, a chamber, and a second specimen outlet were formed in an OCA film, which configures the second base, in the same way as the above.

A first specimen outlet was formed in a PET film, which configures a third base, in the same way as the above.

A third specimen inlet was formed in an OCA film, which configures a fourth base, in the same way as the above.

The specimen inlet and the specimen outlet formed in each of the first to fourth bases are attached and stacked to correspond to each other, thereby biosensors of first and second comparative examples were manufactured.

TABLE 1

| | |
|---|---|
| Moisture absorber A | Thickness(m): 0.000169 |
| (Pore size 11 μm) | Basis weight(kg/m²): 0.089074467 |
| Whatman ® Grade 1 | Density of cellulose: 1540 kg/m³ |
| Qualitative Filter Paper | Porosity: 0.6577 |
| Moisture absorber B | Thickness(m): 0.0001735 |
| (Pore size 8 μm) | Basis weight(kg/m²): 0.100303531 |
| Whatman ® Grade 2 | Density of cellulose: 1540 kg/m³ |
| Qualitative Filter Paper | Porosity: 0.6246 |
| Moisture absorber C | Thickness(m): 0.0002037 |
| (Pore size 20 μm) | Basis weight(kg/m²): 0.088547034 |
| Whatman ® Grade 4 | Density of cellulose: 1540 kg/m³ |
| Qualitative Filter Paper | Porosity: 0.7177 |
| Moisture absorber D | Thickness(m): 0.0001788 |
| (Pore size 3 μm) | Basis weight(kg/m²): 0.098642762 |
| Whatman ® Grade 6 | Density of cellulose: 1540 kg/m³ |
| Qualitative Filter Paper | Porosity: 0.6418 |

TABLE 2

| | Width of | Width of | | | Width of | Width of |
|---|---|---|---|---|---|---|
| | first | second | | | second | first |
| | specimen | specimen | Chamber | Moisture | specimen | specimen |
| Unit: μm | inlet | inlet | height | absorber | outlet | outlet |
| Example 1 | 300 | 400 | 200 | A | 400 | 300 |
| Example 2 | 300 | 200 | 200 | B | 200 | 300 |
| Example 3 | 600 | 600 | 210 | C | 600 | 600 |
| Example 4 | 300 | 300 | 190 | D | 300 | 300 |
| Comparative | 90 | 100 | 100 | — | 100 | 100 |
| example 1 | | | | | | |
| Comparative | 1200 | 300 | 100 | — | 300 | 1200 |
| example 2 | | | | | | |

### Experiment Example

Evaluation 1: Evaluation of Biosensor Based on Moisture Absorber

Whether a specimen was injected, an injection speed of a specimen, the ratio of bubbles produced in flow passages when injection was finished, and a stabilization index were evaluated by injecting a specimen having glucose density of 0.1 mM into the biosensors according to the Examples and the comparative examples, and the results are shown in the following Table 3.

The stabilization index means a value that is calculated by dividing a value, which is obtained by subtracting a current value L measured in a stabilized state from a current value Iₜ measured at specific time, by the current value L measured in a stabilized state, and then multiplying this value by 100.

Since the smaller the stabilization index, the more the precision of a biosensor is improved, which means that the measurement time of biosensors, of which the stabilization indexes measured at the same time are small, of different biosensors is further reduced and the precision thereof is improved.

Stabilization index=[(Iₜ−I₈)/I₉]*100

TABLE 3

| | 30 s | 60 s | | |
|---|---|---|---|---|
| | Stabilization | Stabilization | Injection | Production of |
| | index | index | speed | bubbles (%) |
| Example 1 | 0.79 | 0.17 | high | 0 |
| Example 2 | 0.89 | 0.19 | medium | 0 |
| Example 3 | 0.36 | 0.1 | high | 0 |
| Example 4 | 0.76 | 0.18 | low | 0 |
| Comparative | 2.12 | 0.73 | low | 60 |
| example 1 | | | | |
| Comparative | 2.25 | 0.81 | medium | 80 |
| example 2 | | | | |

Referring to the contents in Table 3, it is possible to see that the 30 s and 60 s stabilization indexes measured by the biosensors of the first to fourth Examples are smaller than the stabilization indexes at the same times measured by the biosensor of the first and second comparative examples.

Further, it is possible to see that according to the biosensors of the first to fourth Examples, no bubble was produced in flow passages when a specimen flowed inside, but, according to the biosensors of the first and second comparative examples, bubbles were produced when a specimen flowed inside.

Therefore, according to the present disclosure, it is possible to manufacture a biosensor of which the specimen inflow speed is good, the measurement time is further reduced, and the precision is further improved, as compared with biosensors of the related art.

Evaluation 2: Evaluation of Biosensor Based on Specimen Density

Stabilization indexes were measured by injecting specimens respectively having glucose density of 0.1 mM, 0.2 mM, and 0.3 mM into the biosensors of the third Example and the first comparative examples, and the results are shown in the following Table 4 and FIG. 7.

TABLE 4

| | 30 s Stabilization index | | | 60 s Stabilization index | | |
|---|---|---|---|---|---|---|
| | 0.1 mM | 0.2 mM | 0.3 mM | 0.1 mM | 0.2 mM | 0.3 mM |
| Example 3 | 0.35 | 0.33 | 0.21 | 0.1 | 0.03 | 0.01 |
| Comparative | 2.12 | 2.38 | 1.61 | 0.73 | 0.79 | 0.6 |
| example 1 | | | | | | |

Referring to the contents in Table 4 and FIG. 7, it is possible to see that, at the glucose density of 0.1 to 0.3 mM, the 30 s and 60 s stabilization indexes measured by the biosensor of the third Example is smaller than the stabilization indexes at the same times measured by the biosensor of the first comparative example.

Therefore, according to the present disclosure, it is possible to manufacture a biosensor of which the measurement time is further reduced and the precision is further improved, as compared with biosensors of the related art, even for various density ranges within a measurement range.

### INDUSTRIAL APPLICABILITY

Since a biosensor according to the present disclosure suppresses bubbles that may be produced in a chamber when a specimen flows inside by having a moisture absorber, which easily absorbs moisture, in a chamber, differences between samples are minimized, whereby it is possible to improve detection precision and reduce a measurement time.

Further, since a biosensor according to the present disclosure can smoothly obtain a specimen even without a specific device by appropriately adjusting the number, width, etc. of bases, specimen inlets, and specimen outlets, it is possible to remove the inconvenience of artificially taking a specimen from an analysis object.

Further, a biosensor according to the present disclosure can continuously measure an analyte included in a specimen by continuous inflow and outflow of a specimen.

1. A biosensor comprising:
a first specimen inlet configured to provide a space through which a specimen flows inside;
an electrode element configured to measure an electromechanical signal of the flowing-in specimen;
a chamber configured to provide a space in which an electrochemical reaction of the flowing-in specimen occurs; and
a first specimen outlet configured to provide a space through which the flowing-in specimen flows outside,
wherein a moisture absorber is disposed in the chamber.

2. The biosensor of claim 1, wherein the moisture absorber has a porosity of 0.5 to 0.8 that is calculated by following Equation 1.
$$ε = 1 - \frac{{bw}_{0}}{{ρ}_{cel} {τ}_{p}} \qquad [ Equation 1 ]$$
in the Equation 1, ε is a porosity of the moisture absorber, bw₀ is basis weight (kg/m²) of the moisture absorber, ρ_{cel} is density of cellulose (kg/m³) of the moisture absorber, and τₚ is a thickness (m) of the moisture absorber.

3. The biosensor of claim 1, wherein a height of the chamber is 50 to 1,000 μm.

4. The biosensor of claim 1, the biosensor has a stacked structure including:
a first base;
a second base formed on the first base; and
a third base formed on the second base.

5. The biosensor of claim 4, wherein the first specimen inlet is disposed in the first base.

6. The biosensor of claim 5, wherein a width of the first specimen inlet is 100 to 1,000 μm.

7. The biosensor of claim 4, wherein the chamber is disposed in the second base.

8. The biosensor of claim 7, wherein the second base further includes:
a second specimen inlet formed at a position corresponding to the first specimen inlet; and
a channel configured to guide a specimen flowing in the second specimen inlet to the chamber.

9. The biosensor of claim 8, wherein a width of the channel is 100 to 1,000 μm.

10. The biosensor of claim 7, wherein the chamber is directly connected to the first specimen inlet.

11. The biosensor of claim 4, wherein the electrode element is disposed between the first base and the second base.

12. The biosensor of claim 4, wherein the first base and the second base each independently include one or more kinds selected from a group of glass, polyethersulfone (PES), poly methyl methacrylate (PMMA), polycarbonate (PC), polyethylene (PE), polyethylene naphthalate (PEN), polyphenylene sulfide (PPS), polypropylene (PP), triacetyl cellulose (TAC), cellulose acetate propionate (CAP), polyethylene terephthalate (PET), polyimide (PI), polyetherimide (PEI), polyamide (PA), cyclo olefin polymer (COP), cyclo olefin copolymer (COC), PMMA/PC copolymer, and PMMA/PC/PMMA copolymer.

13. The biosensor of claim 4, wherein the second base is made of a PSA (pressure sensitive adhesive) compound or an OCA (optical clear adhesive) compound.

14. The biosensor of claim 4, further comprising a fourth base formed under the first base,
wherein the fourth base has a third specimen inlet.

15. The biosensor of claim 4, wherein the first specimen outlet is disposed in the third base.

16. The biosensor of claim 15, wherein a width of the first specimen outlet is 100 to 1,000 μm.
